(19) United States
(12) Patent Application Publication
(10) Pub. No.: US 2026/0294331 A1
(43) Pub. Date: Oct. 1, 2026

(54) AUTOMATED SKIN CONDITION EVALUATION

(71) Applicant: ELI LILLY AND COMPANY, Indianapolis, IN (US)

(72) Inventors: Klaus Theodor GOTTLIEB, Cayucos, CA (US); Derek Thomas ONKEN, Indianapolis, IN (US)

(21) Appl. No.: 19/164,300

(22) PCT Filed: Mar. 25, 2024
(86) PCT No.: PCT/US2024/021294

§ 371 Date: Sep. 11, 2025

Related U.S. Application Data
(60) Provisional application No. 63/493,468, filed on Mar. 31, 2023.

(51) Int. Cl. A61B 5/00; A61B 5/103; A61B 5/107

(52) U.S. Cl. CPC A61B 5/443; A61B 5/0077; A61B 5/1032; A61B 5/1075; A61B 5/7278; A61B 5/7485; A61B 2576/00

(57) ABSTRACT

Aspects of the present disclosure relate to automated skin condition evaluation. A region of interest within image data of a user is identified that is afflicted by a skin or nail condition. A control region that is unafflicted by the condition may similarly be identified, such that a set of chromophore concentrations is generated for the control region. Using the set of chromophore concentrations, an estimated thickness is generated for the region of interest. Thus, it is assumed that the amount of chromophores at the control region are at least approximately the same as the amount of chromophores at the region of interest, such that a difference in coloration between the control region and the region of interest is predominantly the result of a difference in thickness. A skin/nail condition severity metric may thus ultimately be generated for the region of interest using the estimated thickness and/or a surface roughness.

### BACKGROUND

Certain conditions that affect human skin and/or nails may increase the thickness of skin and/or nails. For instance, skin or nail psoriasis may cause hyperkeratosis (excessive thickness of skin tissue), as well as pitting or other visible features on skin or nails. However, objectively characterizing the severity and/or progression of such conditions may be difficult. Similarly, while treatment may be developed and administered to address these and other conditions, objectively evaluating the effect of the treatment on an afflicted region may be challenging.

It is with respect to these and other general considerations that embodiments have been described. Also, although relatively specific problems have been discussed, it should be understood that the embodiments should not be limited to solving the specific problems identified in the background.

### SUMMARY

Aspects of the present disclosure relate to automated skin condition evaluation. In examples, a region of interest within image data of a user is identified that is afflicted by a skin or nail condition. A control region that is not afflicted by the condition may similarly be identified, such that a set of chromophore concentrations is generated for the control region. Using the set of chromophore concentrations, an estimated thickness is generated for the region of interest. Thus, it is assumed that the amount of chromophores at the control region are at least approximately the same as the amount of chromophores at the region of interest, such that a difference in coloration between the control region and the region of interest is predominantly the result of a difference in thickness (since chromophore concentration would be expected to decrease with increasing skin thickness if the amount of chromophores are held constant).

Further, some skin conditions may alter the roughness or texture of the skin without significantly affecting the skin's associated thickness, such that surface roughness may additionally or alternatively be determined for the region of interest. Using an estimated thickness and/or a surface roughness, a skin/nail condition severity metric may be generated for the region of interest. An indication of such thickness, surface roughness, and/or condition severity metrics may ultimately be provided to a user, thereby enabling the objective determination as to whether a skin/nail condition is present. Additionally, or alternatively, it may thus be possible to objectively track the progression of a skin/nail condition over time.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following Figures.

FIG. 1 illustrates an overview of an example system with which automated skin condition evaluation may be performed according to aspects described herein.

FIG. 2A illustrates example image data of a hand of a user that may be evaluated according to the disclosed automated skin condition evaluation techniques.

FIG. 2B illustrates an example control region that is used for automated skin condition evaluation according to aspects described herein.

FIG. 2C illustrates an example region of interest that is evaluated based on the control region of FIG. 2B according to aspects described herein.

FIG. 3A illustrates an overview of an example method for generating an estimated thickness for a region of interest according to aspects described herein.

FIG. 3B illustrates an overview of another example method for generating an estimated thickness for a region of interest according to aspects described herein.

FIG. 4A illustrates an overview of an example method for determining a condition severity based on an estimated thickness and an estimated roughness according to aspects described herein.

FIG. 4B illustrates an overview of an example method for determining whether a skin condition is present according to aspects described herein.

FIG. 5 illustrates an example of a suitable operating environment in which one or more aspects of the present application may be implemented.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Embodiments may be practiced as methods, systems, or devices. Accordingly, embodiments may take the form of a hardware implementation, an entirely software implementation, or an implementation combining software and hardware aspects. The following detailed description is therefore not to be taken in a limiting sense, and the scope of the present disclosure is defined by the appended claims and their equivalents.

Certain conditions that affect human skin and/or nails may increase the thickness of skin and/or nails. As another example, such conditions may cause pitting or other visible features on skin or nails. While treatment may be developed and administered to address these and other conditions, it may be difficult to objectively evaluate the severity/progression of such conditions, as well as the effect of treatment. For example, an individual's skin and/or nails may be manually scored (e.g., by the individual or by a proctor), which may inadvertently introduce inconsistencies across individuals, or even across observations for the same individual, due to the subjectivity of manual scoring.

Accordingly, aspects of the present disclosure relate to automated skin condition evaluation. In examples, image data is captured of a user (e.g., including the user's hands, feet, nails, arms, legs, or any of a variety of other skin and/or nail regions). A region of interest is identified (e.g., manually or automatically) within the image data, where the region of interest is afflicted by a skin or nail condition. In examples, a region that is not afflicted by the skin or nail condition is similarly identified within the image data (which is referred to herein as a “control region”).

A set of chromophore concentrations is generated for the control region based on the image data for the control region. In examples, a model that generates the set of chromophore concentrations based on the image data for the control region uses a predetermined or otherwise assumed tissue (e.g., skin/nail) thickness for the control region. An estimated tissue thickness is then generated for the region of interest based on the set of chromophore concentrations generated for the control region.

This estimate of tissue thickness for the control region may be generated by applying the aforementioned model in reverse. As such, it is assumed that the chromophore concentrations of the control region are applicable to the region of interest, such that a difference in coloration between the control region and the region of interest is predominantly the result of a difference in thickness (as the chromophore concentration for the region of interest would be expected to decrease with increasing skin thickness, assuming the amount of chromophores is substantially similar). It will be appreciated that the resulting estimated thickness for the region of interest may thus at least be a relative measurement (e.g., relative to the control region), which may be used to identify a difference in thickness between the control region and the region of interest, as well as changes in thickness over time, among other examples. Similarly, an indication as to an estimated thickness for a region of interest may be provided as a relative measurement as compared to a control region.

A surface roughness is additionally or alternatively determined for a region of interest. For example, some skin conditions (e.g., prurigo nodularis) may alter the roughness or texture of the skin without significantly affecting the skin's associated thickness. Other conditions may alter both tissue thickness and roughness. As such, evaluating surface roughness in addition to or as an alternative to thickness may enable the evaluation of a wider range of skin/nail conditions.

Using a thickness and/or a surface roughness determined according to aspects described herein, a skin/nail condition severity metric may be generated. For example, the condition severity metric is generated based on a combination of the thickness and the surface roughness for a region of interest. The combination may be generated based on a thickness weighting and a roughness weighting, such that the condition severity metric is a weighted average of the generated thickness and roughness metrics. As another example, the condition severity metric is generated using a machine learning model that is trained (e.g., using annotated training data and/or reinforcement learning) to generate a condition severity metric for a set of inputs that includes a determined thickness and/or surface roughness for a region of interest. As a further example, the condition severity metric may be generated based on multiple thickness and/or surface roughness determinations (e.g., for multiple regions of interest and/or for measurements for the same region of interest at multiple times).

Generated thickness, surface roughness, and/or condition severity metrics may be used for any of a variety of subsequent processing according to aspects described herein. For instance, an indication of one or more such metrics may be provided to a user, thereby enabling the user to determine whether a skin/nail condition is present and/or to track the progression of the condition over time. As another example, such metrics may be generated for a population of users based on image data for each user that was acquired over time, such that the efficacy of a skin/nail treatment may be objectively quantified and evaluated across the population of users.

It will therefore be appreciated that aspects of the present disclosure may be performed using image data that was recently captured (e.g., contemporaneously with the disclosed processing) and/or may be performed retrospectively (e.g., based on image data that was previously captured), among other examples. As another example, a first set of thickness, surface roughness, and/or condition severity metrics may be generated (e.g., contemporaneously with when the image data was captured) and a second set of metrics may be generated for the image data at a later point in time, as may be the case when different or improved models are available with which to process the image data and generate one or more associated metrics.

A set of chromophore concentrations may be generated for a control region and/or a region of interest according to any of a variety of techniques. It will be appreciated that any of a variety of wavelength bands may be used. For instance, spectral reconstruction or spectral super-resolution may be used to reconstruct any number of wavelength bands (e.g., between 400 nm to 700 nm at 10 nm intervals from RGB image data that contains three spectral bands). Additional examples of such aspects are described in the following paper, which is hereby incorporated by reference in its entirety: Kaya, B., Can, Y. B., & Timofte, R. (2018). Towards Spectral Estimation from a Single RGB Image in the Wild. https://doi.org/10.48550/arxiv.1812.00805.

Intensities in the respective wavelength bands (e.g., in the red, green, and blue channels) may correspond to the respective concentrations of various chromophores (e.g., melanin, oxygenated hemoglobin, deoxyhemoglobin, and bilirubin), such that the processed wavelength bands are further processed (e.g., based on an absorption coefficient for each chromophore, which may be associated with a tissue thickness) to generate a set of chromophore concentrations accordingly.

For example, a set of wavelength band intensities (e.g., for red, green and blue channels and/or for bands that were determined using spectral reconstruction or spectral super-resolution) may be multiplied by a color transformation matrix N₁, thereby converting the set of wavelength band intensities into another color system or other set of wavelengths. Said other color system or other set of wavelengths may have any number m of dimensions. As an example, N₁ may be mathematically determined based on one or more known techniques for translating between one color system to another color system. The resulting set of wavelength band intensities is referred to herein as a spectral vector.

In examples, the resulting spectral vector is multiplied by an estimation matrix N₂, thereby generating a vector of chromophore concentrations (also referred to herein as a set of chromophore concentrations) that corresponds to a chromophore space. As an example, the estimation matrix is determined using a computer model of human skin (e.g., comprising the stratum corneum, epidermis, and dermis). In such an example, the computer model may be programmed with absorption coefficients corresponding to various chromophores, as may be obtained from literature. Alternatively, or additionally, N₂ may be determined (and/or validated) using experimental data, among other examples.

Additional examples of such aspects are described by the following paper, which is hereby incorporated by reference in its entirety: Nishidate, I., Minakawa, M., McDuff, D., Wares, M. A., Nakano, K., Haneishi, H., Aizu, Y., & Niizeki, K. (2020). Simple and affordable imaging of multiple physiological parameters with RGB camera-based diffuse reflectance spectroscopy. Biomedical Optics Express, 11(2), 1073-1091. https://doi.org/10.1364/BOE.382270.

As another example, a method for determining whether skin in a region of interest is likely to exhibit abnormal thickness (e.g., as compared to a control region) is provided. In examples, a set of wavelength band intensities represented by the vector C (e.g., according to the RGB color system, [symbol] ³) for the control region is transformed according to a color transformation matrix, thereby yielding a spectral vector X for the control region in a spectral space (e.g., [symbol] ^{m}, having m dimensions that each correspond to a wavelength band). Restated, a function ƒ(C) is used to generate the spectral vector X for a set of wavelength band intensities C for the control region according to color transformation matrix N₁, where ƒ(C)=N₁C. Accordingly, the resulting spectral vector X (e.g., as may be defined as ƒ(C)) is further transformed into a vector Y in the chromophore space (e.g., [symbol] ⁶, having six example chromophores each corresponding to a dimension) based on estimation matrix N₂ by function g(X), where g(X)=N₂X. Methods for determining a suitable estimation matrix N₂ are provided in the aforementioned Nishidate publication. While examples are described with respect to various matrices (which may thus imply linear transformations), it will be appreciated that such aspects are illustrative and, in other examples, nonlinear functions ƒ(C) and g(X) are also possible.

In examples, ƒ(C) and g(X) may each be approximately invertible. For example, if N₁ is a square invertible matrix, the inverse of ƒ(C) may thus be defined as ƒ⁻¹(X)=N₁⁻¹X, thus solving the inverse problem N₁C=X for an unknown C. Similarly, if N₂ is a square invertible matrix, the inverse of g(X) may thus be defined as g⁻¹(Y)=N₂⁻¹Y, thus solving the inverse problem N₂X=Y for an unknown X.

Accordingly, the chromophore vector for the control region, which may be defined as g(ƒ(C))=Y according to the above-described functions, may thus be used to compute an expected set of wavelength band intensities C₂, where C₂=ƒ⁻¹(g⁻¹(Y)). This expected set of wavelength band intensities C₂ represents the wavelengths that we expect to observe from the region of interest if the skin tissue thickness at the region of interest is the same as that of the control region. Accordingly, a set of wavelength band intensities R for a region of interest may thus be compared according to the example equation below:

$$‖ R - C ‖ > λ ‖ {C}_{2} - C ‖$$

Where ∥⋅∥ is a distance metric (e.g., the Euclidean distance between the two vectors R and C) and λ≥1 is a user-defined tuning value. If the above equation is true for wavelength band intensities C of the control region and R of the region of interest, it may be determined that the region of interest exhibits an abnormal skin thickness. In this example, the term ∥C₂−C∥ indicates the amount of error that may be introduced by digital processing of wavelength band intensity data. Such errors may result from the rounding that inevitably results from digitizing analog data wavelength data, and the compounding of such rounding errors through mathematical matrix operations. Such errors are unavoidably introduced by performing any digital mathematical operation on analog wavelength data. As a result, any difference between R and C (∥R−C∥) that is less than or equal to the difference between λ∥C₂−C∥ may be due to errors introduced by said digital processing rather than any true difference in the wavelength band intensities C and R. On the other hand, any difference between R and C that is greater than λ∥C₂−C∥ can no longer be explained by digital processing error and is therefore indicative of actual differences in coloration between the region of interest and the control region. If the above equation is true, then an estimated thickness (t) of tissue at the region of interest as compared to the control region may be quantified according to the example equation below:

$$t = ‖ R - C ‖ > λ ‖ {C}_{2} - C ‖$$

In some embodiments, any differences in coloration between the region of interest and the control region are assumed to be due to thicker skin at the region of interest compared to the control region. Therefore, the thickness t computed by the equation above is an estimate of how much thicker skin at the region of interest is compared to the skin at the control region.

As another example, if ƒ and g are assumed to have no inverse, Y and Z may instead be computed and evaluated, where Y=g(ƒ(C)) and Z=g(ƒ(R)). Thus, similar to the example function above, a distance metric ∥⋅∥ may be used to evaluate a distance between Y and Z, such that if the distance exceeds a tuning value λ≥0, it is determined that the region of interest exhibits an abnormal skin thickness. An example equation of such an analysis is provided below.

$$‖ Y - Z ‖ > λ$$

If the above equation is true, then an estimated thickness (t) of tissue at the region of interest as compared to the control region may be quantified according to the example equation below:

$$t = ‖ Y - Z ‖ > λ$$

Finally, in instances where an evaluation based on the chromophore space according to function g is omitted and function ƒ is again assumed to have no inverse, a similar comparison may be performed in the spectral space based on ƒ(C) and ƒ(R) as described above, for a tuning value λ≥0:

$$‖ f ( C ) - f ( R ) ‖ > λ$$

Thus, similar to the prior two example equations, if the above equation evaluates to true, it may be determined that the region of interest exhibits an abnormal skin thickness. If so, then an estimated thickness (t) of tissue at the region of interest as compared to the control region may be quantified according to the example equation below:

$$t = ‖ f ( C ) - f ( R ) ‖ - λ$$

In some embodiments, any of the aforementioned estimates of thickness t may be further converted by some function h(t) that converts detected differences in coloration between the region of interest and the control region to units of length, such as millimeters or microns. The function h(t) may be experimentally determined and may, in some examples, comprise multiplying the thickness t by an experimentally determined scaling constant.

Similarly, a surface roughness may be determined for a control region and/or a region of interest according to any of a variety of techniques. For example, the image data may be processed to determine a set of excerpts (e.g., cropped segments of the image having a predetermined width and/or according to a predetermined spacing, wherein such cropped segments may partially overlap or not overlap). In examples, the image data is converted to greyscale to reduce the impact of color variations as compared to variability in brightness/darkness. Each of the determined excerpts is processed to evaluate variations in pixel values along the excerpt. As a result, an excerpt that exhibits a higher degree of variability may thus be determined to have more surface roughness than an excerpt that exhibits a lower degree of variability, such that a surface roughness metric may be generated accordingly. Additional examples of such aspects are described by the following paper, which is hereby incorporated by reference in its entirety: Jafari, A., Fazayeli, A., & Zarezadeh, M. (2014). Estimation of orange skin thickness based on visual texture coarseness. Biosystems Engineering, 117, 73-82. https://doi.org/10.1016/j.biosystemseng.2013.08.010.

While various techniques are discussed above with respect to wavelength band processing, tissue thickness estimation, and surface roughness estimation, it will be appreciated that such aspects are provided as examples and, in other examples, any of a variety of additional or alternative techniques may be used.

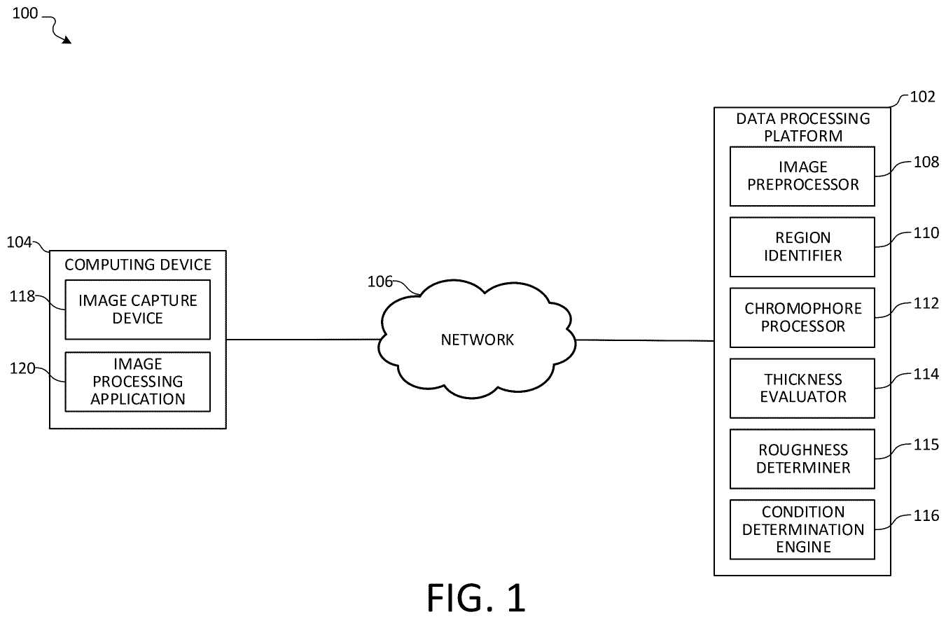

FIG. 1 illustrates an overview of an example system 100 with which automated skin condition evaluation may be performed according to aspects described herein. As illustrated, system 100 comprises data processing platform 102, computing device 104, and network 106. In examples, data processing platform 102 and computing device 104 communicate via network 106. For example, network 106 may comprise a local area network, a wireless network, or the Internet, or any combination thereof, among other examples.

As illustrated, data processing platform 102 includes image preprocessor 108, region identifier 110, chromophore processor 112, thickness evaluator 114, roughness determiner 115, and condition determination engine 116.

In examples, image preprocessor 108 obtains image data from computing device 104, which is preprocessed according to aspects described herein. It will be appreciated that image preprocessor 108 may obtain image data from any of a variety of additional or alternative sources, as may be the case when previously stored image data is processed. In examples, image preprocessor 108 generates image data corresponding to one or more wavelength bands (e.g., a red, green, and blue channel or corresponding to any of a variety of wavelength ranges). As another example, image preprocessor 108 generates a greyscale representation of the image data, as may be used by roughness determiner 115. In other examples, image preprocessor 108 adjusts the brightness and/or contrast of the image data to account for the lighting of the environment in which the image data was obtained (e.g., by image capture device 118). As a further example, image preprocessor 108 generates a spectral vector for a given set of wavelength band intensities, for example based on a color transformation matrix N₁ according to aspects described herein. It will thus be appreciated that any of a variety of preprocessing operations may be performed by image preprocessor 108.

Region identifier 110 processes the image data to identify a region of interest and/or a control region. In examples, region identifier 110 identifies a region based on a user indication received from computing device 104. For example, image processing application 120 may enable a user to identify one or more regions of interest and/or control regions.

As another example, such regions may be automatically identified. For example, region identifier 110 automatically identifies a control region that includes skin and/or nails that are “normal” (e.g., unafflicted by a condition for which the processing is being performed). In examples, the control region is determined based on a region of interest (e.g., as may be automatically or manually determined), as may be the case when a control region is identified at a similar part of the user's body as the region of interest. As a further example, a region of skin or a nail that is relatively smooth may be identified as the control region.

It will thus be appreciated that any of a variety of techniques may be used to identify a control region and/or a region of interest according to aspects described herein. Further, while such aspects are described with respect to region identifier 110 of data processing platform 102, it will be appreciated that similar aspects may be alternatively or additionally implemented by image processing application 120. For instance, image processing application 120 may perform such aspects to extract identified regions and thus reduce the amount of data that is transmitted from computing device 104 to data processing platform 102. It will also be appreciated the order in which image preprocessor 108 and region identifier 110 operates on image data received from the computing device 104 may be interchangeable, according to different embodiments. In some examples, the region identifier 110 may act on image data received from the computing device 104 first, and then the image preprocessor 108 may act on image data output from the region identifier 110. Alternatively, the image preprocessor 108 may act on image data received from the computing device 104 first, and then the region identifier 110 may act on image data output from the image preprocessor 108.

Data processing platform 102 is further illustrated as comprising chromophore processor 112, which processes image data for a control region (e.g., as was identified by region identifier 110 and/or preprocessed by image preprocessor 108) to generate a set of chromophore concentrations according to aspects described herein. As noted above, any of a variety of techniques may be used to generate the set of chromophore concentrations. For example, mathematical transformations N₁ (e.g., a color transformation matrix, to the extent not already applied by image preprocessor 108) and N₂ (e.g., an estimation matrix) may be used to evaluate functions ƒ(C) and g(X) described above, thereby generating a set of chromophores based on a set of wavelength bands for given image data (e.g., corresponding to control region and a region of interest). Example chromophores include, but are not limited to, melanin, oxygenated hemoglobin, deoxyhemoglobin, and bilirubin. It will be appreciated that fewer, additional, or alternative chromophores may be used in other examples.

Thickness evaluator 114 processes a set of chromophore concentrations to generate an estimated thickness corresponding to a region of interest (e.g., as may have been identified by region identifier 110). For example, the set of chromophore concentrations may correspond to a control region and may have been generated by chromophore processor 112. As another example, one or more evaluations according to the example equations described above may be performed. For instance, thickness evaluator 114 evaluates a distance between a set of wavelength band intensities for a control region C and a region of interest R is compared to a distance between C and an expected set of wavelength band intensities C₂ according to tuning value λ (e.g., ∥R−C∥>λ∥C₂−C∥) to determine whether the region of interest exhibits an abnormal skin thickness. In another example, thickness evaluator 114 performs processing according to the above-described example equations ∥Y−Z∥>λ and/or ∥ƒ(C)−ƒ(R)∥>λ, thereby determining whether the region of interest exhibits an abnormal skin thickness. As a further example, a set of chromophores may be determined for a given region of interest, as may be the case when a data store includes known or expected chromophore concentrations for a given demographic and/or skin/nail region, among other examples. Thus, it will be appreciated that a control region need not be used in some examples. In such cases, a set of wavelength band intensities Co that is derived from expected chromophore concentrations for a given demographic and/or skin/nail region (e.g., as experimentally determined or observed from a population of other users) may be substituted in place of the set of wavelength band intensities C from a control region in the aforementioned calculations and equations.

In examples, thickness evaluator 114 processes a pixel of the image data corresponding to the region of interest based on the set of chromophore concentrations to generate a corresponding estimate of thickness t or h(t), as previously explained. In examples, multiple pixels within the region of interest are processed to generate an estimate of thickness for each pixel, thereby forming a thickness map that indicates a thickness gradient for tissue within the region of interest. In other examples, a single estimated thickness (e.g., relative to the control region) may be generated for multiple pixels within the region of interest, or even for the entire region of interest as a whole.

Roughness determiner 115 processes a region of interest to generate a surface roughness metric associated therewith. As noted above, any of a variety of techniques may be used to generate the surface roughness metric. For example, a greyscale representation of the region of interest (e.g., as may have been generated by image preprocessor 108) is processed to generate one or more strips or excerpts therein, such that brightness/darkness variations within a strip or excerpt may be used to generate the surface roughness metric accordingly (e.g., where higher variability is indicative of increased surface roughness).

Data processing platform 102 further comprises condition determination engine 116, which processes an estimated thickness (or a thickness map) generated by thickness evaluator 114 and/or a surface roughness generated by roughness determiner 115 to generate a condition severity metric. As noted above, the severity metric may comprise a weighted average of the estimated thickness and the surface roughness. In other examples, a machine learning model is used to generate a classification based on the estimated thickness and/or surface roughness accordingly. Thus, condition determination engine 116 evaluates a thickness map and/or a surface roughness corresponding to a region of interest to determine a condition and a severity associated therewith.

Data processing platform 102 may provide an indication of a determined thickness, surface roughness, and/or condition severity metric to computing device 104. In other examples, generated metrics and/or associated image data are stored in a data store for subsequent processing. For instance, historical metrics may be maintained for one or more individuals, thereby enabling longitudinal analysis of the progression of an individual's skin/nail condition. It will therefore be appreciated that the data processing techniques described herein may be used in any of a variety of contexts.

As illustrated, computing device 104 comprises image capture device 118 and image processing application 120. In examples, computing device 104 is a mobile computing device, a tablet computing device, or a laptop computing device. Image capture device 118 is usable to capture image data, including, but not limited to, an image and/or one or more frames of video.

As an example, image processing application 120 obtains image data from image capture device 118, which is processed (e.g., by data processing platform 102) according to aspects described herein. As an example, a user operates image processing application 120 to capture at least a portion of an individual's body using image capture device 118. In examples, the individual is the user (e.g., as may be the case if the user is using image processing application 120 for self-diagnosis). As another example, another individual may operate computing device 104 (e.g., as may be the case in a clinical trial).

The captured image data is provided to data processing platform 102, such that one or more generated metrics (e.g., as may be generated by thickness evaluator 114, roughness determiner 115, and/or condition determination engine 116) may be received in response. In examples, data processing platform 102 provides an indication as to an identified skin condition and an associated severity metric. The indication may be presented to the user of computing device 104 by image processing application 120 accordingly.

While computing device 104 is illustrated as comprising image capture device 118, it will be appreciated that, in other examples, image capture device 118 may be another device, such as a camera, which communicates with computing device 104 and/or data processing platform 102 (e.g., via a wired connection, a wireless connection, and/or network 106). Additionally, it will be appreciated that while system 100 is illustrated as comprising one data processing platform 102 and one computing device 104, any number of such elements may be used in other examples. For example, image data from multiple devices may be processed by a single data processing platform or, as another example, different computing devices may each have an associated data processing platform.

Further, it will be appreciated that, in other examples, the functionality described herein may be distributed among or otherwise implemented according to any of a variety of other configurations. For example, computing device 104 may implement aspects associated with data processing platform 102, such that image data obtained by image capture device 118 is processed by image processing application 120 in addition to or as an alternative to processing performed by data processing platform 102. Performing data processing local to computing device 104 may offer improved user privacy and may reduce the amount of data that is transferred to data processing platform 102, among other examples.

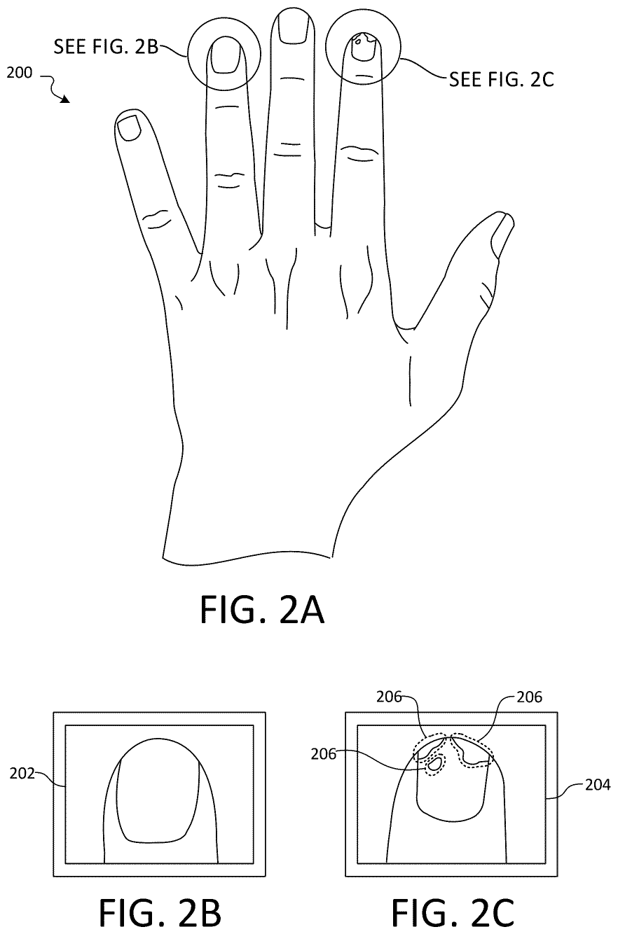

FIG. 2A illustrates example image data 200 of a hand of a user that may be evaluated according to the disclosed automated skin condition evaluation techniques. For instance, image data 200 may be captured by image capture device 118 as a result of a user operating image processing application 120 of computing device 104 in FIG. 1. While image data 200 depicts the hand of an individual, it will be appreciated that any of a variety of other regions of a user's body may be evaluated in other examples.

Image data 200 may be processed according to aspects described herein to identify control region 202 and region of interest 204, as illustrated by FIGS. 2B and 2C. For example, regions 202 and 204 may be automatically identified by a region identifier, such as region identifier 110 discussed above with respect to FIG. 1. As another example, user input may be received (e.g., by an image processing application, such as image processing application 120) that includes an indication of a boundary corresponding to control region 202 and/or a boundary corresponding to region of interest 204. As a further example, user input may indicate a general region of image data 200, such that the general region is processed by a region identifier to identify a more specific control region and/or region of interest therein.

As illustrated, the nail in control region 202 of FIG. 2B is processed according to aspects described herein to generate a set of chromophore concentrations, with which a thickness of the nail in region of interest 204 in FIG. 2C is generated. In examples, a thickness map is generated, which may indicate different (e.g., increased) thicknesses corresponding to regions 206 illustrated in FIG. 2C accordingly. Thus, it may be determined whether the nail affliction illustrated in regions 206 results from differences in nail thickness or from some other condition.

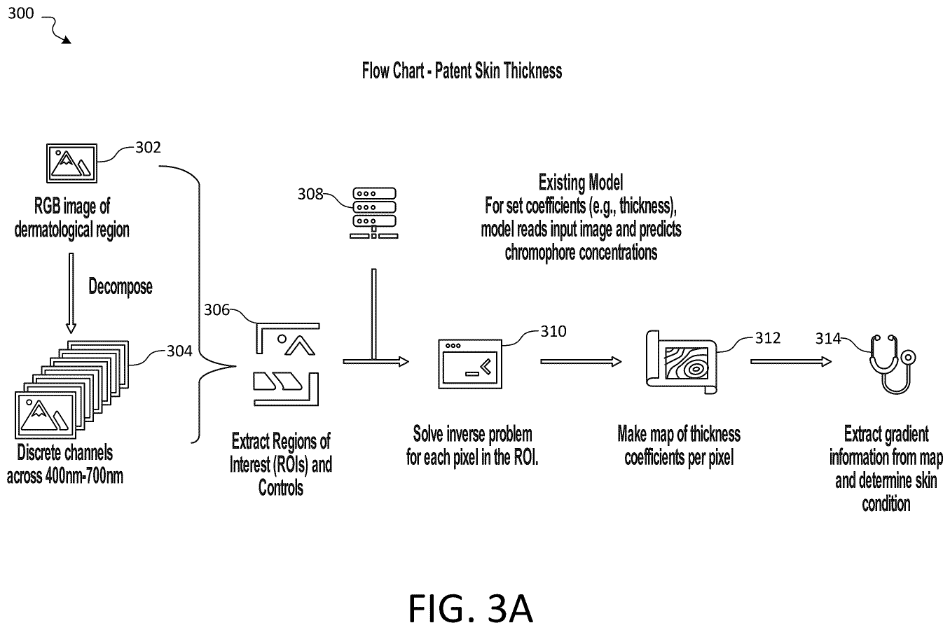

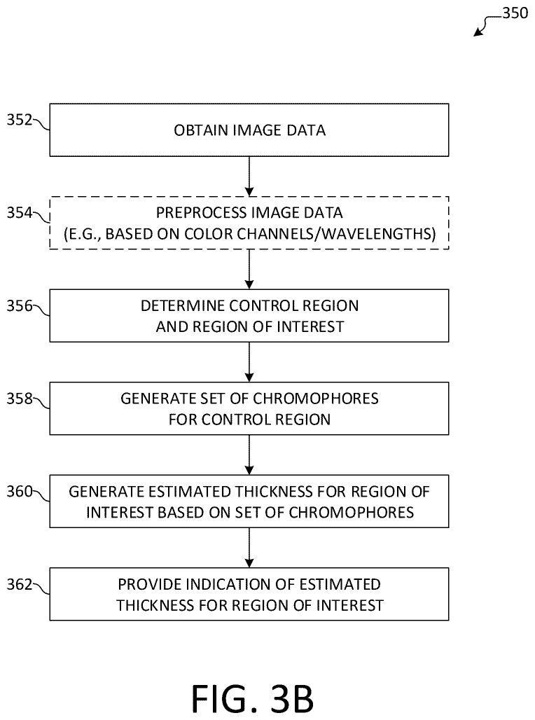

FIG. 3A illustrates an overview of an example method 300 for generating an estimated thickness for a region of interest according to aspects described herein. In examples, aspects of method 300 are performed by a data processing platform, such as data processing platform 102 in FIG. 1.

Method 300 begins at operation 302, where RGB image data of a dermatological region is obtained. For example, the image data is obtained from an image capture device of a computing device, such as image capture device 118 of computing device 104 in FIG. 1. As another example, the image data is obtained from a data store (e.g., of a data processing platform). It will therefore be appreciated that image data may be obtained from any of a variety of sources.

At operation 304, the obtained image data is decomposed into discrete wavelength bands (e.g., between 400 nm and 700 nm, as illustrated). Aspects of operation 304 may be performed by an image preprocessor, such as image preprocessor 108 in FIG. 1. In examples, the discrete wavelength bands include a spectral vector that correspond to a spectral space, as may be generated based on a color transformation matrix (e.g., according to function ƒ(X) and color transformation matrix N₁ described above). Next, at operation 306, one or more regions of interest and control regions are extracted. As noted above, the extracted regions may be identified automatically and/or based on user input (e.g., as may be identified by a region identifier, such as region identifier 110 in FIG. 1).

Method 300 progresses to operation 308, where image data corresponding to the extracted control region(s) is processed to generate a set of chromophore concentrations. As illustrated, a model including a set of coefficients corresponding to a predetermined thickness may be used to generate the set of chromophore concentrations in some examples. Such aspects may be performed by a chromophore processor (e.g., chromophore processor 112 in FIG. 1) in some examples. For instance, an estimation matrix N₂ is used to transform a spectral vector and thus generate the set of chromophores (e.g., according to function g(X) described above).

As a result, one or more pixels in the region(s) of interest that were extracted at operation 306 are processed at operation 310 to generate a corresponding estimated thickness (e.g., the estimated thickness t or h(t), as may be generated by a thickness evaluator, such as thickness evaluator 114 in FIG. 1). As noted above, the set of chromophore concentrations that was generated at operation 308 may be used to determine the estimated thickness, such that the amount of chromophores is assumed to be substantially similar while the thickness is permitted to vary, as the chromophore concentration may decrease with increasing skin thickness (thereby solving the inverse problem as was processed at operation 308). As another example, one or more comparisons are performed at operation 308 (e.g., based on a color transformation matrix and/or an estimation matrix) to determine whether the region of interest exhibits an abnormal skin thickness, as were discussed above with respect to thickness evaluator 114 and the associated example equations.

At operation 312, a thickness map is generated based on the estimated thicknesses (e.g., t or h(t)) that were generated at operation 310. For instance, multiple pixels or other subparts of a region of interest may be processed to generate a corresponding estimated thickness, such that the thickness map generated at operation 312 includes each of the thickness estimates.

Flow progresses to operation 314, where gradient information is extracted from the thickness map and used to determine a condition for the region of interest accordingly. In examples, aspects of operation 314 are performed by a condition determination engine, such as condition determination engine 116 in FIG. 1. Thus, operation 314 may determine an associated condition and/or a condition severity metric, among other examples. In some instances, operation 314 comprises providing an indication of the determined condition and/or severity (e.g., to a computing device, such as computing device 104). As another example, operation 314 comprises storing the generated metrics and/or associated data in a data store for subsequent processing. Method 300 terminates at operation 314.

FIG. 3B illustrates an overview of another example method 350 for generating an estimated thickness for a region of interest according to aspects described herein. In examples, aspects of method 350 are performed by a data processing platform, such as data processing platform 102 in FIG. 1.

Method 350 begins at operation 352, where image data is obtained. For example, image data may be obtained from a data store or from an image capture device (e.g., image capture device 118 of computing device 104 in FIG. 1), among other examples. The image data comprises at least a part of a user's body, such as one or more regions that are afflicted by a skin/nail condition. Aspects of operation 352 may be similar to those discussed above with respect to operation 302 of method 300 in FIG. 3A and are therefore not necessarily redescribed in detail.

Flow progresses to operation 354, where the image data that was obtained at operation 352 is preprocessed. For example, the image data may be decomposed into its constituent red, green, and blue channels. As another example, image data corresponding to one or more wavelength bands is generated. Aspects of operation 354 may be performed by an image preprocessor, such as image preprocessor 108 in FIG. 1. Aspects of operation 354 may be similar to those discussed above with respect to operation 304 of method 300 in FIG. 3A and are therefore not necessarily redescribed in detail.

Operation 354 is illustrated using a dashed box to indicate that, in some examples, operation 354 may be omitted. For instance, the image data obtained at operation 352 may not need to be preprocessed, as may be the case when the image data is captured in controlled or consistent conditions or image data corresponding to one or more wavelength bands is already provided, among other examples.

At operation 356, a control region and a region of interest are determined. Aspects of operation 356 may be similar to those discussed above with respect to operation 306 of method 300 in FIG. 3A and are therefore not necessarily redescribed in detail. For instance, the regions may be automatically identified or may be determined based on a received user indication (e.g., corresponding to user input at an application, such as image processing application 120), among other examples. In some instances, aspects of operation 356 are performed by a region identifier, such as region identifier 110 in FIG. 1.

Flow progresses to operation 358, where a set of chromophores are generated for the control region that was determined at operation 356. Aspects of operation 358 may be similar to those discussed above with respect to operation 308 of method 300 in FIG. 3A and are therefore not necessarily redescribed in detail. For instance, aspects of operation 358 may be performed by a chromophore processor, such as chromophore processor 112 in FIG. 1. Operation 358 may comprise determining a set of chromophore concentrations that contribute to or otherwise explain a coloration depicted within image data corresponding to the control region according to any of a variety of techniques as described above.

At operation 360, an estimated thickness is generated for image data corresponding to a region of interest based on the set of chromophore concentrations that was generated at operation 358. Aspects of operation 360 may be similar to those discussed above with respect to operation 310 and/or 312 of method 300 in FIG. 3A and are therefore not necessarily redescribed in detail. In examples, multiple thickness estimates are generated (e.g., for multiple pixels or other subparts within the region of interest). In such examples, a thickness map including the thickness estimates may be generated accordingly, thereby indicating a thickness gradient within the region of interest. As another example, the resulting estimate includes an indication as to whether the region of interest exhibits an abnormal skin thickness, which may thus be a binary indication.

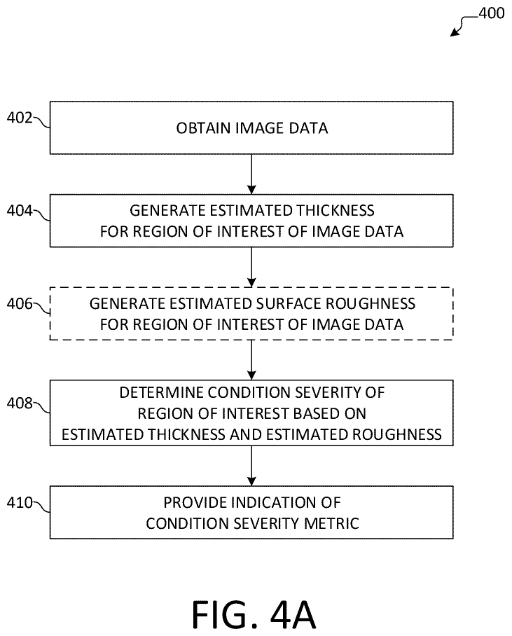

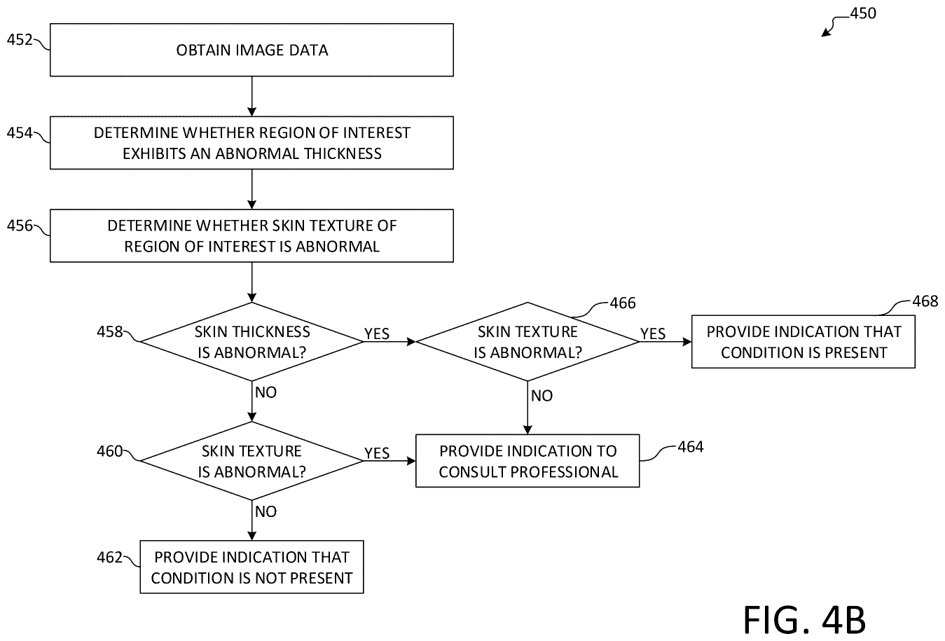

Moving to operation 362, an indication of the estimated thickness(es) for the region of interest is provided. For instance, the indication may be provided to a computing device, such as computing device 104 in FIG. 1. Alternatively, or additionally, an indication is provided as to whether the region of interest exhibits an abnormal skin thickness. In other examples, the indication is stored in a data store for subsequent evaluation, as may be the case when the progression of a corresponding condition is analyzed over time. As a further example, the indication may be provided for subsequent processing to generate a condition severity metric according to aspects described herein, as may be generated by a condition determination engine (e.g., condition determination engine 116 in FIG. 1) performing aspects of method 400 in FIG. 4A, which is discussed in greater detail below. Method 350 terminates at operation 362. In another example, the indication is provided for subsequent processing to determine whether a skin condition is present according to aspects described herein, as may be generated by performing aspects of method 450 in FIG. 4B.

FIG. 4A illustrates an overview of an example method 400 for determining a condition severity based on an estimated thickness and an estimated roughness according to aspects described herein. In examples, aspects of method 400 are performed by a data processing platform, such as data processing platform 102 in FIG. 1.

Method 400 begins at operation 402, where image data is obtained. For example, image data may be obtained from a data store or from an image capture device (e.g., image capture device 118 of computing device 104 in FIG. 1), among other examples. The image data comprises at least a part of a user's body, such as one or more regions that are afflicted by a skin/nail condition. Aspects of operation 402 may be similar to those discussed above with respect to operations 302 and 352 of methods 300 and 350, respectively, and are therefore not necessarily redescribed in detail.

At operation 404, an estimated thickness is generated for a region of interest of the image data that was obtained at operation 402. Operation 404 may comprise performing aspects of method 300 and/or 350 discussed above with respect to FIGS. 3A and 3B, respectively.

Flow progresses to operation 406, where an estimated surface roughness is generated. Aspects of operation 406 may be performed by a roughness determiner, such as roughness determiner 115 in FIG. 1. As noted above, any of a variety of techniques may be used to generate the surface roughness metric. For example, a greyscale representation of the region of interest (e.g., as may have been generated by an image preprocessor, such as image preprocessor 108) is processed at operation 406 to generate one or more strips therein, such that brightness/darkness variations therein may be used to generate the surface roughness metric accordingly.

Operation 406 is illustrated using a dashed box to indicate that, in some examples, operation 406 may be omitted. For example, a condition severity metric may be generated based on an estimated thickness and an estimated surface roughness (e.g., in instances where operations 404 and 406 are performed) or based on an estimated thickness (e.g., in instances where operation 406 is omitted), among other examples.

Moving to operation 408, a condition severity corresponding to the region of interest is determined based on the estimated thickness that was generated at operation 404 and, optionally, the estimated surface roughness that was generated at operation 406. Aspects operation 408 may be performed by a condition determination engine, such as condition determination engine 116 in FIG. 1. Aspects of operation 408 may be similar to those discussed above with respect to operations 314 of FIG. 3A and are therefore not necessarily redescribed below in detail.

For instance, the severity metric may comprise a weighted average of the estimated thickness and the surface roughness. In other examples, a machine learning model is used to generate a classification based on the estimated thickness and/or surface roughness accordingly. Thus, it will be appreciated that any of a variety of techniques may be used to generate a condition severity metric based on an estimated thickness and/or a surface roughness according to aspects described herein.

At operation 410, an indication of the condition severity metric is provided. For instance, the indication may be provided to a computing device, such as computing device 104 in FIG. 1. In other examples, the indication is stored in a data store for subsequent evaluation, as may be the case when the progression of a corresponding condition is analyzed over time. Method 400 terminates at operation 410.

FIG. 4B illustrates an overview of an example method 450 for determining whether a skin condition is present according to aspects described herein. In examples, aspects of method 400 are performed by a data processing platform, such as data processing platform 102 in FIG. 1.

Method 450 begins at operation 452, where image data is obtained. For example, image data may be obtained from a data store or from an image capture device (e.g., image capture device 118 of computing device 104 in FIG. 1), among other examples. The image data comprises at least a part of a user's body, such as one or more regions that are afflicted by a skin/nail condition. Aspects of operation 452 may be similar to those discussed above with respect to operations 302, 352, and/or 402 of methods 300, 350, and 400, respectively, and are therefore not necessarily redescribed in detail.

At operation 454, it is determined whether a region of interest of the image data exhibits an abnormal thickness. Aspects of operation 454 may be performed by a thickness evaluator, such as thickness evaluator 114 discussed above with respect to FIG. 1. As noted above, any of a variety of techniques may be used to determine whether the region of interest exhibits an abnormal thickness. As an example, one or more of the above-described equations are used to process image data corresponding to the region of interest and a control region.

Flow progresses to operation 456, where an estimated surface roughness is generated. Aspects of operation 406 may be performed by a roughness determiner, such as roughness determiner 115 in FIG. 1. As noted above, any of a variety of techniques may be used to generate the surface roughness metric. For example, a greyscale representation of the region of interest (e.g., as may have been generated by an image preprocessor, such as image preprocessor 108) is processed at operation 406 to generate one or more strips therein, such that brightness/darkness variations therein may be used to generate the surface roughness metric accordingly.

At determination 458, it is determined whether the skin thickness is abnormal. For example, the determination may comprise determining whether processing that was performed at operation 454 yielded a true evaluation processing result. If it is determined that the skin thickness is not abnormal, flow branches “NO” to determination 460, where it is determined whether the skin texture is abnormal. If it is determined that the skin texture is not abnormal, flow branches “NO” to operation 462, where an indication is provided (e.g., to a computing device, such as computing device 104 in FIG. 1) that a skin condition is likely not present. It will be appreciated that any of a variety of additional or alternative operations may be performed, for example to store the indication in association with the image data, thereby enabling historical or retrospective analysis of an individual's skin. Method 450 terminates at operation 462.

Returning to determination 460, if it is instead determined that the skin texture is abnormal, flow branches “YES” to operation 464, where an indication is provided (e.g., to a computing device, such as computing device 104 in FIG. 1) to consult a professional. Thus, method 450 may yield an indeterminate result in instances where the skin thickness is not abnormal (e.g., as a result of branching “NO” at determination 458) and the skin exhibits an abnormal texture (e.g., branching “YES” at determination 460). Similar to operation 462, any of a variety of additional or alternative processing may be performed at operation 464. Method 450 terminates at operation 464.

Returning to determination 458, if it is instead determined that the skin thickness is abnormal, flow branches “YES” to determination 466, where it is determined whether the skin texture is abnormal. Aspects of determination 466 may be similar to those discussed above with respect to operation 460 and are therefore not redescribed in detail. Accordingly, if it is determined that the skin texture is not abnormal, flow branches “NO” to operation 464, which was discussed above. Thus, method 450 may yield an indeterminate result in instances where the skin thickness is abnormal (e.g., as a result of branching “YES” at determination 458) and the skin does not exhibit an abnormal texture (e.g., branching “NO” at determination 466).

Returning to determination 466, if it is instead determined that the skin texture is abnormal, flow branches “YES” to operation 468, where an indication is provided (e.g., to a computing device, such as computing device 104 in FIG. 1) that a skin condition is likely present. It will be appreciated that any of a variety of additional or alternative operations may be performed, for example to store the indication in association with the image data, thereby enabling historical or retrospective analysis of an individual's skin. Method 450 terminates at operation 466.

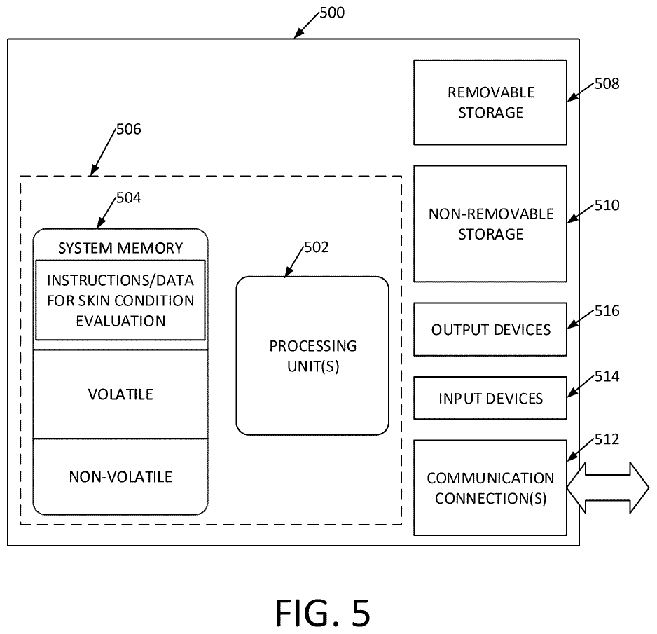

FIG. 5 illustrates an example of a suitable operating environment 500 in which one or more of the present embodiments may be implemented. This is only one example of a suitable operating environment and is not intended to suggest any limitation as to the scope of use or functionality. Other well-known computing systems, environments, and/or configurations that may be suitable for use include, but are not limited to, personal computers, server computers, hand-held or laptop devices, multiprocessor systems, microprocessor-based systems, programmable consumer electronics such as smart phones, network PCs, minicomputers, mainframe computers, distributed computing environments that include any of the above systems or devices, and the like.

In its most basic configuration, operating environment 500 typically may include at least one processing unit 502 and memory 504. Depending on the exact configuration and type of computing device, memory 504 (storing, among other things, APIs, programs, etc. and/or other components or instructions to implement or perform the system and methods disclosed herein, etc.) may be volatile (such as RAM), non-volatile (such as ROM, flash memory, etc.), or some combination of the two. This most basic configuration is illustrated in FIG. 5 by dashed line 506. Further, environment 500 may also include storage devices (removable, 508, and/or non-removable, 510) including, but not limited to, magnetic or optical disks or tape. Similarly, environment 500 may also have input device(s) 514 such as a keyboard, mouse, pen, voice input, etc. and/or output device(s) 516 such as a display, speakers, printer, etc. Also included in the environment may be one or more communication connections, 512, such as LAN, WAN, point to point, etc.

Operating environment 500 may include at least some form of computer readable media. The computer readable media may be any available media that can be accessed by processing unit 502 or other devices comprising the operating environment. For example, the computer readable media may include computer storage media and communication media. The computer storage media may include volatile and nonvolatile, removable and non-removable media implemented in any method or technology for storage of information such as computer readable instructions, data structures, program modules, or other data. The computer storage media may include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transitory medium, which can be used to store the desired information. The computer storage media may not include communication media.

The communication media may embody computer readable instructions, data structures, program modules, or other data in a modulated data signal such as a carrier wave or other transport mechanism and includes any information delivery media. The term “modulated data signal” may mean a signal that has one or more of its characteristics set or changed in such a manner as to encode information in the signal. For example, the communication media may include a wired media such as a wired network or direct-wired connection, and wireless media such as acoustic, RF, infrared and other wireless media. Combinations of the any of the above should also be included within the scope of computer readable media.

The operating environment 500 may be a single computer operating in a networked environment using logical connections to one or more remote computers. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet.

The different aspects described herein may be employed using software, hardware, or a combination of software and hardware to implement and perform the systems and methods disclosed herein. Although specific devices have been recited throughout the disclosure as performing specific functions, one skilled in the art will appreciate that these devices are provided for illustrative purposes, and other devices may be employed to perform the functionality disclosed herein without departing from the scope of the disclosure.

As stated above, a number of program modules and data files may be stored in the system memory 504. While executing on the processing unit 502, program modules (e.g., applications, Input/Output (I/O) management, and other utilities) may perform processes including, but not limited to, one or more of the stages of the operational methods described herein such as the methods illustrated in FIG. 2, 3A-3B, or 4A-4B, for example.

Furthermore, examples of the invention may be practiced in an electrical circuit comprising discrete electronic elements, packaged or integrated electronic chips containing logic gates, a circuit utilizing a microprocessor, or on a single chip containing electronic elements or microprocessors. For example, examples of the invention may be practiced via a system-on-a-chip (SOC) where each or many of the components illustrated in FIG. 5 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality described herein may be operated via application-specific logic integrated with other components of the operating environment 500 on the single integrated circuit (chip). Examples of the present disclosure may also be practiced using other technologies capable of performing logical operations such as, for example, AND, OR, and NOT, including but not limited to mechanical, optical, fluidic, and quantum technologies. In addition, examples of the invention may be practiced within a general purpose computer or in any other circuits or systems.

Aspects of the present disclosure, for example, are described above with reference to block diagrams and/or operational illustrations of methods, systems, and computer program products according to aspects of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

1. A system comprising:
at least one processor; and
memory storing instructions that, when executed by the at least one processor, causes the system to perform a set of operations, the set of operations comprising:
obtaining:
image data representative of a control region of skin of a user; and
image data of a region of interest of the skin;
generating, based on the image data representative of the control region, a set of chromophore concentrations;
generating, based on the set of chromophore concentrations and the image data of the region of interest, an estimated skin thickness for the skin in the region of interest; and
providing an indication of the estimated skin thickness.

2. The system of claim 1, wherein:
the set of operations further comprises preprocessing the image data representative of the control region to generate a set of wavelength band intensities for the control region; and
the set of chromophore concentrations is generated based on the set of wavelength band intensities.

3. The system of claim 2, wherein the estimated skin thickness is generated by:
generating an expected set of wavelength band intensities based on the set of chromophore concentrations;
calculating a first value based on a difference between the expected set of wavelength band intensities and the set of wavelength band intensities for the control region;
calculating a second value based on a difference between a set of wavelength band intensities for the region of interest and the set of wavelength band intensities for the control region; and
performing a comparison between the first value and the second value.

4. The system of claim 3, wherein the first value is calculated by multiplying the difference between the expected set of wavelength band intensities and the set of wavelength band intensities for the control region by a tuning factor.

5. The system of claim 3, wherein the estimated skin thickness is generated by calculating a difference between the first value and the second value.

6. (canceled)

7. (canceled)

8. The system of claim 1, wherein the set of operations further comprises receiving user input comprising an indication of the control region and the region of interest.

9. (canceled)

10. The system of claim 1, wherein at least one of the image data representative of the control region or the image data of the region of interest is obtained from an image capture device of the system.

11. The system of claim 1, wherein at least one of the image data representative of the control region or the image data of the region of interest is obtained from a data store.

12. The system of claim 1, wherein:
the set of chromophore concentrations is generated by processing the image data representative of the control region using an estimation matrix to output an estimated concentration of one or more chromophores.

13. The system of claim 1, wherein the set of chromophore concentrations includes a concentration for one or more chromophores selected from a group of chromophores consisting of:
melanin;
bilirubin;
oxygenated blood; and
deoxygenated blood.

14. The system of claim 1, wherein the indication of the estimated skin thickness includes a thickness for the region of interest that is relative to the control region.

15. (canceled)

16. The system of claim 1, wherein the image data representative of the control region comprises data derived from image data of control regions of skin of other users that are different from the user.

17. A method for generating a skin condition severity based on image data, the method comprising:
obtaining image data representative of a control region of skin of a user;
obtaining image data of a region of interest of the skin;
generating, based on the image data representative of the control region, a set of chromophore concentrations;
generating, based on the set of chromophore concentrations and the image data of the region of interest, an estimated skin thickness for the skin in the region of interest; and
providing an indication of the estimated skin thickness.

18. The method of claim 17, wherein:
the method further comprises preprocessing the image data representative of the control region to generate a set of wavelength band intensities for the control region; and
the set of chromophore concentrations is generated based on the set of wavelength band intensities.

19. The method of claim 18, wherein the estimated skin thickness is generated by:
generating an expected set of wavelength band intensities based on the set of chromophore concentrations;
calculating a first value based on a difference between the expected set of wavelength band intensities and the set of wavelength band intensities for the control region;
calculating a second value based on a difference between a set of wavelength band intensities for the region of interest and the set of wavelength band intensities for the control region; and
performing a comparison between the first value and the second value.

20. The method of claim 19, wherein the first value is calculated by multiplying the difference between the expected set of wavelength band intensities and the set of wavelength band intensities for the control region by a tuning factor.

21. The method of claim 19, wherein the estimated skin thickness is generated by calculating a difference between the first value and the second value.

22. (canceled)

23. (canceled)

24. The method of claim 17, wherein the method further comprises receiving user input comprising an indication of the control region and the region of interest.

25. (canceled)

26. The method of claim 17, wherein the set of chromophore concentrations is generated by processing the image data of the control region using an estimation function to output an estimated concentration of one or more chromophores.

27. (canceled)

28. The method of claim 17, wherein the image data representative of the control region comprises data derived from image data of control regions of skin of other users that are different from the user.
